(19) United States
(12) Patent Application Publication
(10) Pub. No.: US 2020/0000386 A1
(43) Pub. Date: Jan. 2, 2020

(54) APPARATUS AND METHODS FOR ANALYTE SENSOR SPATIAL MISMATCH MITIGATION AND CORRECTION

(71) Applicant: GlySens Incorporated, San Diego, CA (US)

(72) Inventors: Piyush Gupta, San Diego, CA (US); Lev Kurbanyan, Granada Hills, CA (US); Joseph Lucisano, San Diego, CA (US); Krista Bertsch, San Diego, CA (US)

(21) Appl. No.: 16/453,794

(22) Filed: Jun. 26, 2019

Related U.S. Application Data
(60) Provisional application No. 62/690,745, filed on Jun. 27, 2018.

(51) Int. Cl. A61B 5/1486; A61B 5/145; A61B 5/1495; A61B 5/00

(52) U.S. Cl. CPC A61B 5/14865; A61B 5/14532; A61B 5/0031; A61B 5/1495; A61B 5/14542

(57) ABSTRACT

Apparatus and methods for reducing error due to spatial arrangement of sensor elements in a parameter sensor such as a physiologic analyte sensor. In one exemplary embodiment, the analyte sensor is configured to measure an analyte of a living being (e.g., blood glucose), and the apparatus and methods employ determination of a blood analyte concentration based on a prescribed relationship of N₁/N₂—i.e., N1 analyte modulated sensing elements (e.g., glucose electrodes) associated with and proximate to N₂ background sensing elements of the sensor—in order to compensate for response differences due to spatial arrangement of the sensor elements or “spatial mismatch.” This configuration of sensor elements and method of determining blood analyte concentration (based on multiple background signal electrodes) enables increased accuracy of the sensor.

### PRIORITY AND RELATED APPLICATIONS

This application claims priority to co-owned and co-pending U.S. Provisional Patent Application No. 62/690,745 filed on Jun. 27, 2018 and entitled “Apparatus and Methods for Analyte Sensor Spatial Mismatch Correction,” which is incorporated herein by reference in its entirety.

This application is additionally related to co-owned and co-pending U.S. patent application Ser. No. 15/853,574 filed Dec. 22, 2017 and entitled “Analyte Sensor and Medicant Delivery Data Evaluation and Error Reduction Apparatus and Methods”; Ser. No. 13/559,475 filed Jul. 26, 2012 entitled “Tissue Implantable Sensor With Hermetically Sealed Housing”; Ser. No. 14/982,346 filed Dec. 29, 2015 and entitled “Implantable Sensor Apparatus and Methods”; Ser. No. 15/170,571 filed Jun. 1, 2016 and entitled “Biocompatible Implantable Sensor Apparatus and Methods”; Ser. No. 15/197,104 filed Jun. 29, 2016 and entitled “Bio-adaptable Implantable Sensor Apparatus and Methods”; Ser. No. 15/359,406 filed Nov. 22, 2016 and entitled “Heterogeneous Analyte Sensor Apparatus and Methods”, Ser. No. 15/368,436 filed Dec. 2, 2016 and entitled “Analyte Sensor Receiver Apparatus and Methods”; Ser. No. 15/472,091 filed Mar. 28, 2017 and entitled “Analyte Sensor User Interface Apparatus and Methods”; Ser. No. 15/645,913 filed Jul. 10, 2017 and entitled “Analyte Sensor Data Evaluation and Error Reduction Apparatus and Methods”; Ser. No. 15/853,574 filed on Dec. 22, 2017 and entitled “Analyte Sensor and Medicant Delivery Data Evaluation and Error Reduction Apparatus and Methods”; Ser. No. 16/233,536 filed Dec. 27, 2018 and entitled “Apparatus and Methods for Analyte Sensor Mismatch Correction”; and Ser. No. 16/443,684 filed on Jun. 17, 2019 and entitled “Analyte Sensor Apparatus and Methods,” each of which is incorporated herein by reference in its entirety.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### 1. TECHNICAL FIELD

The disclosure relates generally to the field of data analysis and processing, including for sensors, therapy devices, implants and other devices (such as those which can be used consistent with human beings or other living entities), and in one exemplary aspect to methods and apparatus for accurately measuring physiological parameters such as blood analyte level, through use of a sensor face comprising an enhanced spatial arrangement for differential sensor elements, as well as error identification, analysis, and/or mitigation or correction routines or computer programs, the latter which, inter alia, improve the accuracy and reliability of such physiological parameter measurements.

### 2. DESCRIPTION OF RELATED TECHNOLOGY

Implantable electronics is a rapidly expanding discipline within the medical arts. Owing in part to significant advances in electronics and wireless technology integration, miniaturization, performance, and material biocompatibility, sensors or other types of electronics which once were beyond the realm of reasonable use within a living subject (i.e., in vivo) can now be surgically implanted within such subjects with minimal effect on the recipient subject, and in fact convey many inherent benefits.

One particular area of note relates to blood analyte monitoring for subjects, such as for example glucose monitoring for those with so-called “type 1” or “type 2” diabetes. As is well known, regulation of blood glucose is impaired in people with diabetes by: (1) the inability of the pancreas to adequately produce the glucose-regulating hormone insulin; (2) the insensitivity of various tissues that use insulin to take up glucose; or (3) a combination of these phenomena. Safe and effective correction of this dysregulation often requires daily (if not continuous) blood glucose monitoring.

Currently, glucose monitoring in the diabetic population is based largely on collecting blood by “fingersticking” and determining its glucose concentration by conventional assay. This procedure has several disadvantages, including: (1) the discomfort associated with the procedure, which should be performed repeatedly each day; (2) the near impossibility of sufficiently frequent sampling (some blood glucose excursions require sampling every 20 minutes, or more frequently, to accurately treat); and (3) the requirement that the user initiate blood collection, which precludes warning strategies that rely on automatic early detection. Using the extant fingersticking procedure, the frequent sampling regimen that would be most medically beneficial cannot be realistically expected of even the most committed patients, and automatic sampling, which would be especially useful during periods of sleep, is not available.

Implantable glucose sensors (e.g., continuous glucose monitoring (CGM) sensors) have long been considered as an alternative to intermittent monitoring of blood glucose levels by the fingerstick method of sample collection. These devices may be fully implanted, where all components of the sensor system reside within the body and there are no through-the-skin (i.e. percutaneous) elements, or they may be partially implanted, where certain components reside within the body but are physically connected to additional components external to the body via one or more percutaneous elements, or they may be of a hybrid nature, where one element of the sensor system is fully implanted, but another element required for operation (e.g. a power source for the implanted element) is worn in close proximity to the implanted element, but outside of the body (e.g., on the skin) for the system to operate. Further, such devices provide users a great deal of freedom from potentially painful intermittent sampling methods such as “fingersticking.” as well as having to remember and obtain self-administered blood analyte readings.

The accuracy of blood analyte detection and measurement is an important consideration for implanted analyte sensors, especially in the context of current blood glucose monitoring systems (such as e.g., fully implanted blood glucose sensor systems), and even more so in the future development of implantable blood glucose monitoring systems (such as e.g., in support of the development of an artificial pancreas). Hence, ensuring accurate measurement for extended periods of time (and minimizing the need for any other confirmatory or similar analyses) is of great significance. In conventional sensors, accuracy can be adversely affected by a myriad of factors such as e.g., random noise, foreign body response (FBR), other tissue responses, anoxia or hypoxia in the region of the analyte sensor, blood analyte tissue dynamics, mechanical jarring, migration, and/or other variables.

Exemplary sensors (such as those of the ICGM® system manufactured by the Assignee hereof) utilize a differential (ratiometric) measurement-based sensor for estimating tissue/blood glucose. As with typical differential or ratiometric measurement-based systems, the common mode signal measured in the sensors (electrodes) should be highly correlated (precise), and temporally synchronous to minimize noise/errors, and to allow correct extraction of the true differential component of the signal. For example, if two steady-state-calibrated sensors contain uncorrelated spikes/signals while measuring a common time-varying signal (as shown in FIG. 1), the ratiometric result (ratio of the two measurements) of the sensors will not remain unified, but will instead consist of an error pertaining to the uncorrelated signal, as shown in FIG. 2. Similarly, the difference between the two signals will not remain at zero, but will instead consist of an error, as shown in FIG. 3.

Specifically, FIG. 1 illustrates common-mode signals (such as e.g., background oxygen) measured by two sensors (i.e., Signal 1 from Sensor 1, and Signal 2 from Sensor 2) at a steady state. In FIG. 2, the solid line curve represents the ratio of the measurements from Sensor 1 and Sensor 2 from FIG. 1, while responding to the same time-varying signal. The expected ideal ratiometric measurement (ratio of Signal 1 to Signal 2) as a function of time is unity (shown as dashed line 202 in FIG. 2), whereas the deviation 204 from the dashed line 202 of FIG. 2 is due to the measurement mismatch reflected in FIG. 1.

Similarly, the difference between the two signals will not remain at zero over time, but will instead consist of an error due to response mismatch, as shown in FIG. 3. Specifically, in FIG. 3, the difference in the measurements of Sensor 1 and Sensor 2 while measuring the common mode signal (such as e.g., background oxygen) is shown; the expected ideal differential measurement is 0 (dashed line 302), whereas the deviation 304 from the ideal line 302 is due to the temporal (response) mismatch.

In certain cases, the differences in the measurements of signals (during operation of the sensors) may at least in part be caused by the spatial distribution of the individual sensor elements within one or more dimensions of a sensor face. For instance, individual sensor elements may be disposed at different locations of tissue having different oxygen concentration (partial pressure) patterns, the patterns of which are dependent on the specific tissue microvasculature. For example, the two sensing elements of a differential pair (i.e., reference and analyte-modulated sensing elements) in an exemplary ICGM sensor are subject to two different processes or functions, measuring background oxygen in one and glucose-modulated oxygen in the other, and differences (error) in the estimation of the background oxygen from the reference electrode can increase as a function of the spatial distance between the two elements.

One exemplary configuration of an ICGM sensor design is depicted in FIG. 4. As indicated therein, each of the electrodes 1, 3, 5, and 7 measures a glucose-modulated oxygen signal (labeled “G”) whereas electrodes 2, 4, 6, and 8 measures reference background oxygen (undisturbed or relatively less disturbed by the presence of glucose—labeled “O”).

FIG. 5 shows correlation in pO2 measured between oxygen electrodes in the exemplary ICGM configuration of FIG. 4. A distance between two adjacent electrodes (e.g., a distance between electrode 1 and electrode 2) is utilized as the unit for specifying the distance from the primary electrode. As can be seen in FIG. 5, the correlation in pO2 measured between farther electrodes (pO2ᵢ and pO2ᵢ₊₄, where “i” is any given reference electrode [2,4,6,8]) is lower than the correlation in pO2 measured between nearer electrodes (pO2ᵢ and pO2ᵢ₊₂, where “i” is again any given reference electrode [2,4,6,8]). Thus, in vivo measurements through the multiple oxygen sensing electrodes incorporated in the exemplary ICGM sensor (of FIG. 4) indicates that the correlation between oxygen signals is a function (inversely related) of the physical distance between these sensor electrodes.

Another exemplary configuration of an ICGM sensor design is depicted in FIG. 6. As distinguished from the configuration of FIG. 4, just two of the electrodes (i.e., electrodes 1 and 3) measure a glucose-modulated oxygen signal, whereas each of electrodes 2, 4, 6, and 8, as well as electrodes 5 and 7, measures reference background oxygen. This particular arrangement of oxygen electrodes shows further characterization of the extent of pO2 variability with respect to spatial distances.

An example of the pO2 measured by two adjacent reference electrodes in vivo is shown in FIG. 7. As can be seen in FIG. 7, the relationship between pO2 measured by the two adjacent electrodes (e.g., 5 and 6) is nonlinear and non-stationary, with long periods of time where pO2 measured by electrode 5 (measured estimate) was either lower or higher than that measured by electrode 6 (neighboring estimate). Thus, in certain cases the discrepancy (differences) in two common-mode signals may not remain constant over time and/or under different operating conditions, and may change as a function of time, spatial arrangement, fluctuations in tissue characteristics, and/or other parameters.

Accordingly, there is a salient need for, inter alia, methods and apparatus which compensate for or at least partly correct for mismatch between, for example, differentially paired electrodes due to their spatial arrangement. Ideally, such methods and apparatus would enable enhanced correlation and precision between measurement of signals; e.g., the background oxygen (common-mode) signal and the glucose-modulated oxygen signal, thereby enabling accurate calculation of blood analyte (e.g., glucose) concentration or other blood analyte data.

### SUMMARY

The present disclosure satisfies the foregoing needs by providing, inter alia, improved methods and apparatus for compensation and mismatch mitigation within a physiological parameter sensor, such as e.g., a differential oxygen-based blood glucose sensor.

In a first aspect, a sensor face apparatus for use in an implantable sensor apparatus is disclosed. In one embodiment, the apparatus includes one or more differential sensor groups configured to measure or detect an analyte, and computerized logic configured to determine blood analyte data based on signals received from the one or more differential sensor groups. Further, each of the one or more differential sensor groups has a prescribed relationship or ratio (N₁/N₂) of analyte sensing electrodes (N₁) and associated background electrodes (N₂), such as 1/4. In one variant, the N₂ (e.g., 4) background electrodes are disposed proximate a single analyte-modulated sensing electrode within the sensor face apparatus. In another variant, the background electrodes and the analyte-modulated sensing electrode are disposed within a common seat structure of the sensor face apparatus.

In one implementation, the sensor group(s) is/are part of an implanted blood glucose sensor (i.e., part of a so-called “continuous glucose monitor” or CGM), and the blood analyte data are blood glucose concentration data (and/or corrected blood glucose rate of change (ROC) data). The glucose sensor in this implementation is an oxygen-based glucose sensor.

In another implementation, the glucose sensor is a hydrogen peroxide-based glucose sensor.

In yet another implementation, the glucose sensor includes both a hydrogen peroxide-based sensor and oxygen-based glucose sensor.

In another aspect, a method of operating an implanted analyte sensor is disclosed. In one embodiment, the analyte sensor is configured to determine a concentration of a physiologic analyte, and the method includes: determining a response of an analyte-modulated sensing electrode of the sensor; determining a response of two or more background electrodes of the analyte sensor, the two or more background electrodes disposed proximate to the analyte sensing electrode (in e.g., a “clustered” configuration); and utilizing (i) the determined response of the analyte-modulated sensing electrode, and (ii) the determined response of the two or more background electrodes to determine the concentration of the physiologic analyte or other analyte data (such as e.g., rate of change (ROC)).

In one variant, the physiologic analyte comprises blood glucose, the analyte sensor comprises an oxygen-based blood glucose sensor. In the foregoing variant, the response of the analyte sensing electrode comprises a glucose-modulated oxygen signal, and the response of two or more background electrodes comprises a background oxygen signal.

In one implementation, the two or more background electrodes are configured to produce a combined or composite response. In an alternate implementation, the two or more background electrodes are configured to each produce an individual response, and the method further comprises determining an average the individual responses of each of the two or more background electrodes.

In yet another aspect of the disclosure, a method of monitoring a blood analyte level of a living being using a blood analyte sensing apparatus is disclosed. In one embodiment, the method includes: utilizing a first electrode having an enzyme-embedded membrane overlaid thereon to generate a first signal; utilizing two or more second electrodes having a non-enzyme embedded membrane overlaid thereon to generate a second signal; utilizing the first signal and the second signal to generate blood analyte data.

In one implementation, the enzyme-embedded membrane comprises a cross-linked protein material having glucose oxidase (GOX) and catalase co-immobilized therein, and the non-enzyme membrane comprises a material having a greater oxygen permeability than that of the cross-linked protein material. In one variant, the method further includes applying a temporal mismatch correction to one or more of the first signal or the second signal to correct for delay and/or lag between the first electrode and the two or more second electrodes. In another variant, the method further includes applying a cross-talk calibration value to at least the second signal.

In another aspect, a computer readable apparatus is disclosed. In one embodiment, the computer readable apparatus comprises a storage medium (e.g., magnetic, solid state, optical, or other storage medium) having at least one computer program disposed thereon and readable by a computerized apparatus. The at least one computer program includes, in one variant, a plurality of instructions which, when executed on the computerized apparatus, cause collection of detected response signals from one or more analyte detector groups in order to determine a blood analyte concentration, each of the one or more analyte detector groups comprising at least an analyte sensing electrode associated with and proximate to two or more background signal electrodes.

In another aspect, a method of monitoring a blood analyte level of a living being using a blood analyte sensing apparatus is disclosed.

In yet another aspect, a method of compensating for errors caused at least in part due to spatial proximity of analyte sensing and background signal electrodes is disclosed.

In still another aspect of the disclosure, a portable electronic apparatus is disclosed. In one embodiment, the portable electronic apparatus includes a portable receiver device configured to perform data calculation and transformation operations for an internal (implanted) blood analyte sensor apparatus with which it is in data communication; e.g., via wireless interface.

In a further aspect, an integrated circuit (IC) apparatus is disclosed, In one embodiment, the IC apparatus comprises a digital processor comprising logic stored in a memory device which implements one or more of the foregoing methods. In one variant, the IC apparatus is configured for use within a small form-factor implantable blood analyte monitoring device.

Other features and advantages of the present disclosure will immediately be recognized by persons of ordinary skill in the art with reference to the attached drawings and detailed description of exemplary embodiments as given below.

### BRIEF DESCRIPTION OF THE DRAWINGS

FIG. 1 is a graphical representation of contemporaneous signals measured by prior art sensing apparatus (including Signal 1 from a first sensor element and Signal 2 from a second sensor element, the sensor elements disposed at different locations within a sensing region of the sensing apparatus).

FIG. 2 is a graphical representation of the ratio of the measurements from the prior art sensing apparatus of FIG. 1.

FIG. 3 is a graphical representation of the difference of the measurements from the prior art sensing apparatus of FIG. 1.

FIG. 4 is a schematic diagram of a first exemplary configuration of a sensor face of the prior art sensing apparatus of FIG. 1.

FIG. 5 is a graphical representation of a distance from a primary electrode and correlation coefficients (indicating error) for the reference electrodes within the first exemplary configuration of a sensor face of FIG. 4.

FIG. 6 is a schematic diagram of a second exemplary configuration of a sensor face of the prior art sensing apparatus of FIG. 1.

FIG. 7 is a graphical representation of reference pO2 readings over time for two neighboring or adjacent reference sensor elements within the second exemplary configuration of a sensor face of FIG. 6.

FIG. 8A is a top plan view of an exemplary implantable sensor apparatus, according to one embodiment of the present disclosure.

FIG. 8B is a top plan view of an exemplary configuration of a sensor face of the implantable sensor apparatus of FIG. 8A, according to one embodiment of the present disclosure.

FIG. 8C is a top plan view of an exemplary configuration of a sensor element group (including multiple reference electrodes associated with a single working electrode) of the sensor face of FIG. 8B, according to one embodiment of the present disclosure.

FIG. 8D is a top plan view of the exemplary configuration of a sensor group of FIG. 8B including an exemplary membrane structure overlaid thereon, according to one embodiment of the present disclosure.

FIG. 9A is a perspective view of the exemplary membrane structure of FIG. 8C.

FIG. 9B is a cross-sectional view of the exemplary membrane structure of FIG. 8C.

FIG. 10 is a logical flow diagram representing an exemplary generalized method for sensor apparatus operation according to one embodiment of the present disclosure.

FIG. 11 is a graphical representation of an exemplary algorithmic transformation useful with the various aspects of the present disclosure, the algorithmic transformation configured to transform a signal from Sensor 2 (e.g., one or more background species sensing elements), so as to temporally match a signal of Sensor 1 (e.g., an analyte-modulated sensing element) to correct for or mitigate delay and lag of Sensor 1.

FIG. 12 is a functional block diagram illustrating the exemplary implantable sensor apparatus and a receiver apparatus useful with the various aspects of the present disclosure.

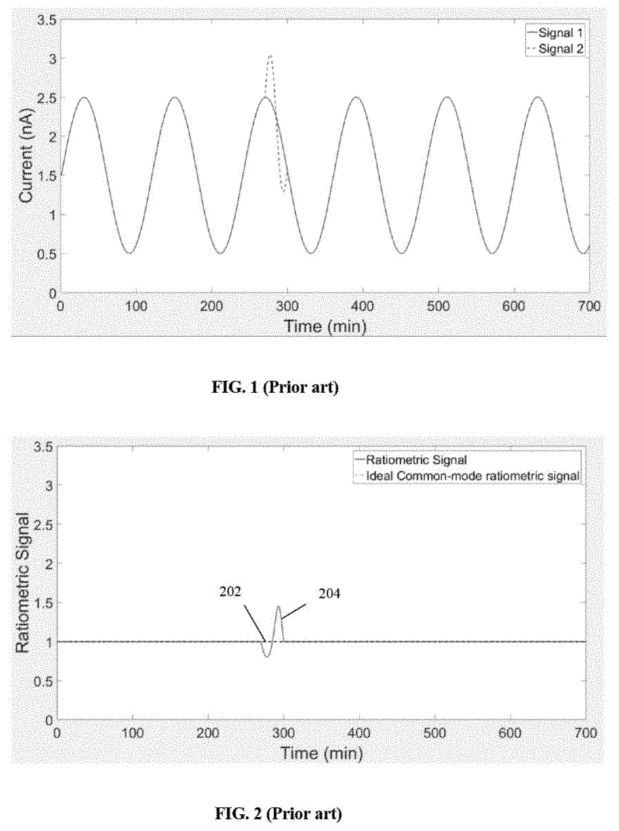

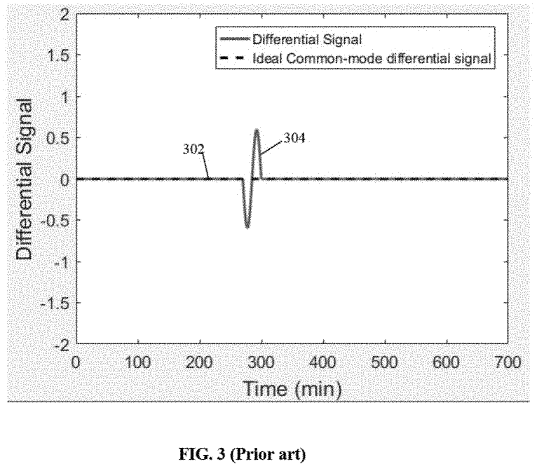

All Figures © Copyright 2017-2019 GlySens Incorporated. All rights reserved.

### DETAILED DESCRIPTION

Reference is now made to the drawings, wherein like numerals refer to like parts throughout.

### Overview

In one exemplary aspect, the present disclosure provides apparatus and methods which at least partly mitigate or compensate for signal error (mismatch) due to spatial arrangements of sensing elements, such as differential background and analyte-modulated sensing elements used in blood analyte detection. In one embodiment, the sensing elements are part of an implantable blood glucose monitor utilizing oxygen-based sensing, and the signal mismatch found in prior art sensors (due to spatial arrangement between background oxygen and glucose-modulated oxygen sensing elements) is reduced or eliminated.

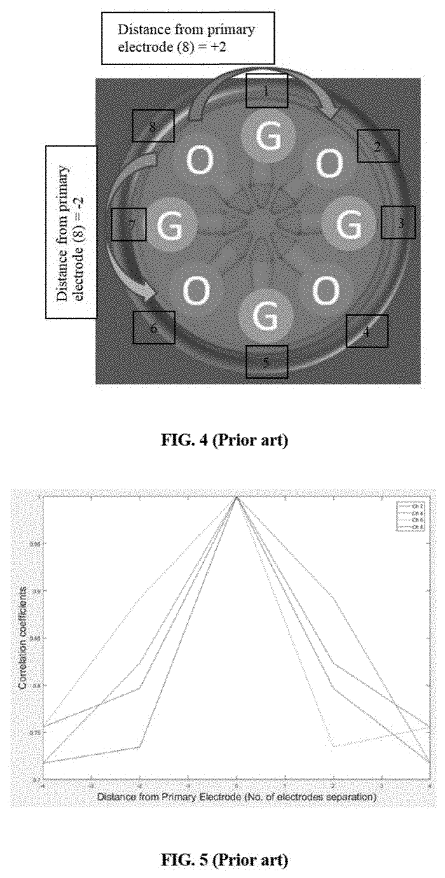

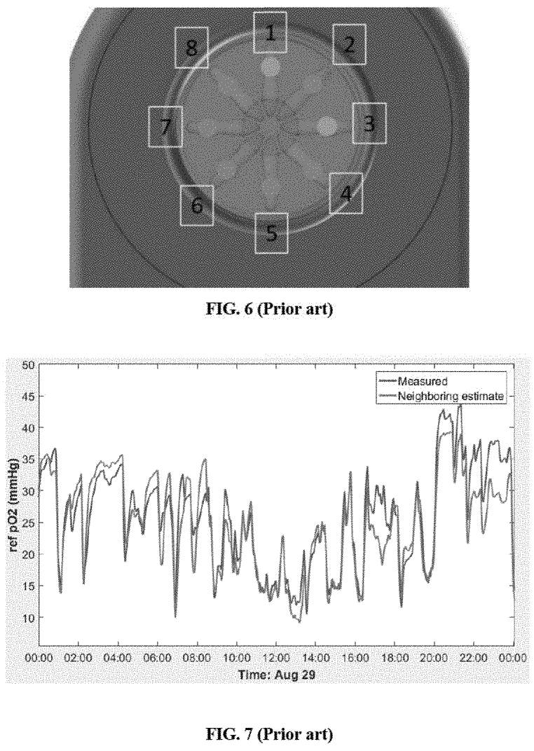

In one implementation, a sensor face includes one or more differential sensing element groups, within which the sensing elements thereof are disposed in a spatially optimized arrangement as compared to those of known or prior art sensors (see e.g., FIGS. 4 and 6). Specifically, each group comprises multiple background sensing elements (e.g., oxygen reference sensing elements) that are spatially arranged to be proximate to an analyte-modulated sensing element (e.g., a glucose-modulated oxygen sensing element). The associated background sensing elements within the group are collectively (and/or selectively) utilized to generate a background oxygen signal via e.g., generation of single signal from all elements within the group, averaging of their individual signals, or other methodology (e.g., various weighting assigned to respective ones of the background sensing elements, selection of signals from one or more background detectors and exclusion of others, etc.).

In one variant, each of the analyte-modulated sensing element includes a working electrode, a reference electrode, and a counter electrode, while the background sensing elements each comprise at least a working electrode and a counter electrode. In one example, the background sensing elements further include a shared reference electrode (i.e., a reference electrode tethered to each of working electrode of the group of background sensing elements). In another example, each of the background sensing elements comprises a separate reference electrode (i.e., an individual reference electrode). Advantageously, the working electrode of the analyte-modulated sensing element is centrally disposed relative to the working electrodes of the background sensing elements in a clustered or otherwise close-proximity spatial arrangement. In order to accommodate the clustered arrangement of the working electrodes, the counter electrodes may be oriented towards a periphery of the sensing element group.

In another implementation, the sensor face further comprises one or more membrane structures associated with the differential sensing element groups. In one variant, the analyte-modulated working electrode and the background working electrodes of each sensing element group are overlaid with a common/unitary membrane structure. The membrane structure includes at least (i) a silicone (or the like) ring or disc which is overlaid on a face of each of the working electrodes of the background sensing elements, and (ii) a central chamber having immobilized enzymes disposed therein and an aperture communicative with an external environment of the sensor face. The central chamber is overlaid on a face of the analyte-modulated working electrode.

In other variants, the common membrane structure covers two or more of the sensing element groups, and has multiple chambers (e.g., comprising chambers having enzymatic material disposed therein overlying each analyte-modulated working electrode for differential sensor groups), or one or more of the sensing elements within a differential group has a separate membrane structure associated therewith (e.g., a first membrane structure overlying the analyte-modulated sensing element and a second membrane structure overlying the background species sensing elements).

Exemplary methods of operating the disclosed sensor apparatus and signal processing are described herein.

The apparatus and methods described herein may also advantageously be applied to similarly benefit other measurement systems that utilize differential, interacting, or otherwise dependent detector or sensor approaches.

### DETAILED DESCRIPTION OF EXEMPLARY EMBODIMENTS

Exemplary embodiments of the present disclosure are now described in detail. While these embodiments are primarily discussed in the context of a fully implantable glucose sensor, such as those exemplary embodiments described herein, and/or those set forth in U.S. Patent Application Publication No. 2013/0197332 filed Jul. 26, 2012 entitled “Tissue Implantable Sensor With Hermetically Sealed Housing”; U.S. Pat. No. 7,894,870 to Lucisano et al. issued Feb. 22, 2011 and entitled “Hermetic Implantable Sensor”; U.S. Patent Application Publication No. 2011/0137142 to Lucisano et al. published Jun. 9, 2011 and entitled “Hermetic Implantable Sensor”; U.S. Pat. No. 8,763,245 to Lucisano et al. issued Jul. 1, 2014 and entitled “Hermetic Feedthrough Assembly for Ceramic Body”; U.S. Patent Application Publication No. 2014/0309510 to Lucisano et al. published Oct. 16, 2014 and entitled “Hermetic Feedthrough Assembly for Ceramic Body”; U.S. Pat. No. 7,248,912 to Gough et al. issued Jul. 24, 2007 and entitled “Tissue Implantable Sensors for Measurement of Blood Solutes”; and U.S. Pat. No. 7,871,456 to Gough et al. issued Jan. 18, 2011 and entitled “Membranes with Controlled Permeability to Polar and Apolar Molecules in Solution and Methods of Making Same”; and U.S. Patent Application Publication No. 2013/0197332 to Lucisano et al. published Aug. 1, 2013 and entitled “Tissue Implantable Sensor with Hermetically Sealed Housing”; PCT Patent Application Publication No. 2013/016573 to Lucisano et al. published Jan. 31, 2013 and entitled “Tissue Implantable Sensor with Hermetically Sealed Housing,” each of the foregoing incorporated herein by reference in its entirety, as well as U.S. patent application Ser. Nos. 15/197,104; 15/359,406; 15/368,436; 15/472,091; 15/645,913; 15/853,574; 16/233,536; and Ser. No. 16/443,684,” each previously incorporated herein, it will be recognized by those of ordinary skill that the present disclosure is not so limited. In fact, the various aspects of the disclosure are useful with, inter alia, other types of sensors, medicant delivery devices having sensing elements, and/or electronic devices, including those relating to other analytes.

Further, while the following embodiments describe specific implementations of e.g., biocompatible oxygen-based multi-sensor element devices for measurement of glucose having specific configurations, protocols, locations, and orientations for implantation (e.g., sensor implantation proximate the waistline on a human abdomen with the sensor array disposed proximate to facial tissue); see e.g., U.S. patent application Ser. No. 14/982,346, entitled “Implantable Sensor Apparatus and Methods” and filed Dec. 29, 2015, previously incorporated herein; those of ordinary skill in the related arts will readily appreciate that such descriptions are purely illustrative, and in fact the methods and apparatus described herein can be used consistent with, and without limitation: (i) in living beings other than humans; (ii) other types or configurations of sensors (e.g., other types, enzymes, and/or theories of operation of glucose sensors, sensors other than glucose sensors, such as e.g., sensors for other analytes such as urea, lactate); (iii) other implantation locations and/or techniques (including without limitation transcutaneous or non-implanted devices as applicable); (iv) other types of differential sensing devices (regardless of the measured analyte or substance), and/or (v) other devices (e.g., non-sensors and non-substance delivery devices).

As used herein, the term “analyte” refers without limitation to a substance or chemical species that is of interest in an analytical procedure. In general, the analyte itself may or may not be directly measurable; in cases where it is not, a measurement of the analyte (e.g., glucose) can be derived through measurement of chemical constituents, components, or reaction byproducts associated with the analyte (e.g., hydrogen peroxide, oxygen, free electrons, etc.). It will be appreciated that although reference is made throughout to “blood analyte,” “blood analyte level,” “blood analyte concentration” or “blood analyte rate of change (ROC)” that the principles of the invention are not restricted to systems where the parameter is solely a blood analyte level. To that end, the terms “blood analyte,” “blood analyte level,” “blood analyte concentration” or “blood analyte rate of change (ROC)” can be taken as synonymous with “physiologic parameter,” “physiologic parameter level,” “physiologic parameter concentration” or “physiologic parameter ROC.”

As used herein, the terms “detector” and “sensor” refer without limitation to a device having one or more elements (e.g., detector element, sensor element, sensing elements, etc.) that generate, or can be made to generate, a signal indicative of a measured parameter, such as the concentration of an analyte (e.g., glucose) or its associated chemical constituents and/or byproducts (e.g., hydrogen peroxide, oxygen, free electrons, etc.). Such a device may be based on electrochemical, electrical, optical, mechanical, thermal, or other principles as generally known in the art. Such a device may consist of one or more components, including for example, one, two, three, or four electrodes, and may further incorporate immobilized enzymes or other biological or physical components, such as membranes, to provide or enhance sensitivity or specificity for the analyte.

As used herein, the term “interface” refers to any signal or data interface with a component or network including, without limitation, those of the FireWire (e.g., FW400, FW800, etc.), USB (e.g., USB 2.0, 3.0. OTG), Ethernet (e.g., 10/100, 10/100/1000 (Gigabit Ethernet), 10-Gig-E, etc.), MoCA, LTE/LTE-A, 5G NR, Wi-Fi (802.11), WiMAX (802.16), Z-wave, PAN (e.g., 802.15)/Zigbee, CBRS (Citizens Broadband Radio Service), Bluetooth, Bluetooth Low Energy (BLE) or power line carrier (PLC) families.

As used herein, the term “computer program” or “software” is meant to include any sequence or human or machine cognizable steps which perform a function. Such program may be rendered in virtually any programming language or environment including, for example, C/C++, Fortran, COBOL, PASCAL, assembly language, markup languages (e.g., HTML, SGML, XML, VoXML), and the like, as well as object-oriented environments such as the Common Object Request Broker Architecture (CORBA), Java® (including J2ME, Java Beans, etc.) and the like.

As used herein, the term “memory” includes any type of integrated circuit or other storage device adapted for storing digital data including, without limitation, ROM, PROM, EEPROM, DRAM, SDRAM, DDR/2 SDRAM, EDO/FPMS, RLDRAM, SRAM, “flash” memory (e.g., NAND/NOR), 3D memory, and PSRAM.

As used herein the terms “parent device” and “parent platform” refers without limitation to any device, group of devices, and/or processes with which a client or peer device (including for example the various embodiments of receiver described in the aforementioned patent applications previously incorporated herein) may logically and/or physically communicate to transfer or exchange data. Examples of parent devices or platforms can include, without limitation, smartphones, tablet computers, laptops, smart watches, personal computers/desktops, servers (local or remote), gateways, dedicated or proprietary analyte receiver devices, medical diagnostic equipment, and even other local receivers acting in a peer-to-peer or dualistic (e.g., master/slave computer architecture) modality.

As used herein, the terms “processor” or “digital processor” are meant generally to include all types of digital processing devices including, without limitation, digital signal processors (DSPs), reduced instruction set computers (RISC), general-purpose (CISC) processors, microprocessors, gate arrays (e.g., FPGAs), PLDs, state machines, reconfigurable computer fabrics (RCFs), array processors, secure microprocessors, and application-specific integrated circuits (ASICs). Such digital processors may be contained on a single unitary integrated circuit (IC) die, or distributed across multiple components.

As used herein, the term “storage” refers to without limitation computer hard drives, memory, RAID devices or arrays, optical media (e.g., CD-ROMs, Laserdiscs, Blu-Ray, etc.), solid state devices (SSDs), flash drives, cloud-hosted storage, or network attached storage (NAS), or any other devices or media capable of storing data or other information.

As used herein, the term “wireless” means any wireless signal, data, communication, or other interface including without limitation Wi-Fi, Bluetooth (including BLE or “Bluetooth Smart”), 3G (3GPP/3GPP2), HSDPA/HSUPA, TDMA, CDMA (e.g., IS-95A, WCDMA, etc.), FHSS, DSSS, GSM, PAN/802.15, WiMAX (802.16), 802.20, Zigbee®, Z-wave, narrowband/FDMA, OFDM, PCS/DCS, LTE/LTE-A/LTE-U/LTE-LAA, 3GPP 5G NR (“New Radio”), CBRS (Citizens Broadband Radio Service), analog cellular, CDPD, satellite systems, millimeter wave or microwave systems, acoustic, and infrared (i.e., IrDA).

### Sensor Face Configuration

As noted above, one aspect of the present disclosure comprises a configuration for sensing elements disposed in a sensing region of a sensor apparatus. The configuration advantageously reduces error in common-mode (background oxygen) signals due to spatial arrangement of the background and analyte-modulated sensing elements, and thereby increases overall accuracy of the sensor.

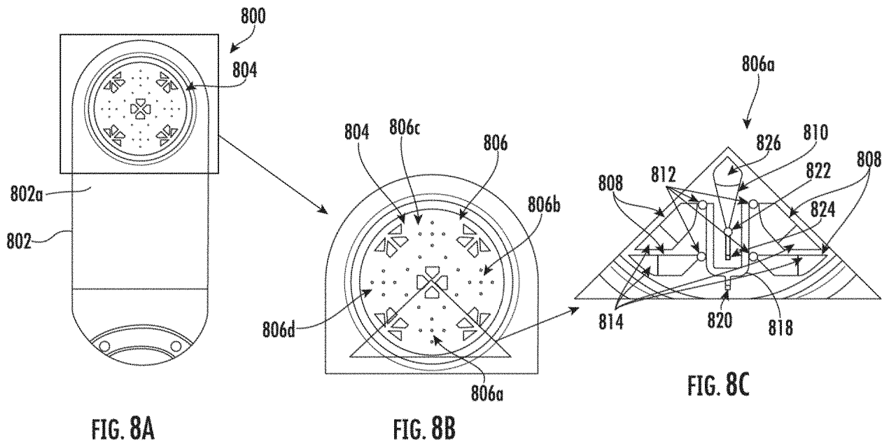

One exemplary embodiment of a fully implantable sensor apparatus 800 having a sensor face 804 is depicted in FIGS. 8A-8D. As can be seen in FIG. 8A, the exemplary sensor apparatus 800 comprises a housing 802 having a sensor face 804 (sensing region) disposed on a top surface 802a thereof. The sensor face 804 includes four groups of sensing elements radially arranged thereon, one of which, sensing element group 806a, is highlighted in FIG. 8B and shown in greater detail in FIGS. 8C and 8D. As can be seen in FIG. 8C, the group of sensing elements 806a includes multiple background species sensing elements 808 (e.g., four background oxygen elements) associated with and proximate to a single analyte-modulated sensing element 810 (e.g., one glucose-modulated oxygen element) for an enhanced spatial relationship of background species and analyte-modulated signals. In alternate embodiments, the sensor face may in include additional or fewer groups of sensors, and/or additional or fewer background (oxygen) elements associated with each analyte-modulated (glucose) element. Additionally, in the present embodiment, each of the sensor element groups has a configuration (discussed infra) which is substantially similar to other sensor groups; however, in alternate embodiments, the sensor elements within each group may have a different configuration/arrangement than that of the other groups (e.g., group 806b having a different configuration than group 806a).

Returning to the embodiment of FIG. 8C, the four background oxygen elements 808 each include a background oxygen (BO) working electrode 812 associated with a BO counter electrode 814. In the exemplary configuration of FIG. 8C, the BO counter electrodes 814 are substantially disposed at opposing lateral sides (proximate to an outer perimeter) of the sensing element group. The orientation of the BO counter electrodes toward the outer perimeter of the sensing element group enables a closer arrangement of the BO working electrodes to the glucose-modulated sensing element. Specifically, the BO working electrodes 812 are evenly-spaced and arranged around a glucose-modulated (GM) working electrode 822 (discussed infra) in a substantially square-shaped and clustered configuration.

In the embodiment depicted in FIG. 8C, each of the BO working electrodes 822 is disposed on a U-shaped filament 818, which is configured for association of each of the BO working electrodes to a single (shared) BO reference electrode 820. The BO reference electrode 820 is proximate to the outer perimeter of the sensor group and an outer perimeter of the sensor face. In alternate embodiments, each of the BO working electrodes may be associated with a separate BO reference electrode. In such embodiments, where each background oxygen electrode has a separate (individual) reference electrode, the U-shaped filament may be excluded. It will be appreciated that utilization of a shared BO reference electrode may enable a reduced size of the sensor face, while individual (separate) BO reference electrodes may enable increased accuracy and/or signal stability for each of the background oxygen elements.

Also shown in FIG. 8C, the glucose-modulated sensing element 810 comprises the GM working electrode 822 (discussed supra), a GM reference electrode 824, and a GM counter electrode 826. The GM electrodes are substantially linearly arranged, where the GM counter electrode 826 is disposed proximate to a center of the sensor face, the GM reference electrode 824 is disposed proximate to the BO reference electrode 820, and the GM working electrode 822 is disposed therebetween (i.e., between the GM counter and reference electrodes). Further, in the illustrated embodiment, the GM working electrode 822 and the GM reference electrode 824 are disposed between the arms of the U-shaped filament 818 of the background oxygen elements 808, while the GM counter electrode 826 is outside of the filament 818.

Similar to the orientation of the BO counter electrodes, the exemplary arrangement of the GM counter electrode enables a “closer” spatial arrangement or proximity of the GM working electrode to the BO working electrodes. As but one example, in a typical spatial arrangement of known sensors, a GM working electrode to a BO working electrode are disposed with a distance of 117 mils therebetween, whereas, in an example of the present spatial arrangement described herein, the GM working electrode and each of the BO working electrodes are disposed with a distance of 68 mils therebetween.

It will be appreciated that the counter electrodes are arranged such that the current path between the working and counter electrodes can be well controlled. The size of the counter electrodes are maximized to ensure enough surface area to service the current needs of the working electrodes so as to e.g., not limit the reaction occurring at the working electrode. The blade-like shape of the counter electrodes is driven by the size constraints of the overall disk, the need for dielectric between channels, and/or a desired distance away from the BO working electrodes. In one variant, both the working and counter electrodes are manufactured from pure platinum. In one implementation the electrode surfaces are further platinized to deposit platinum black on the electrodes to improve performance via e.g., increasing an active surface area. In another implementation, the reference electrodes are pure platinum electroplated to silver/silver chloride.

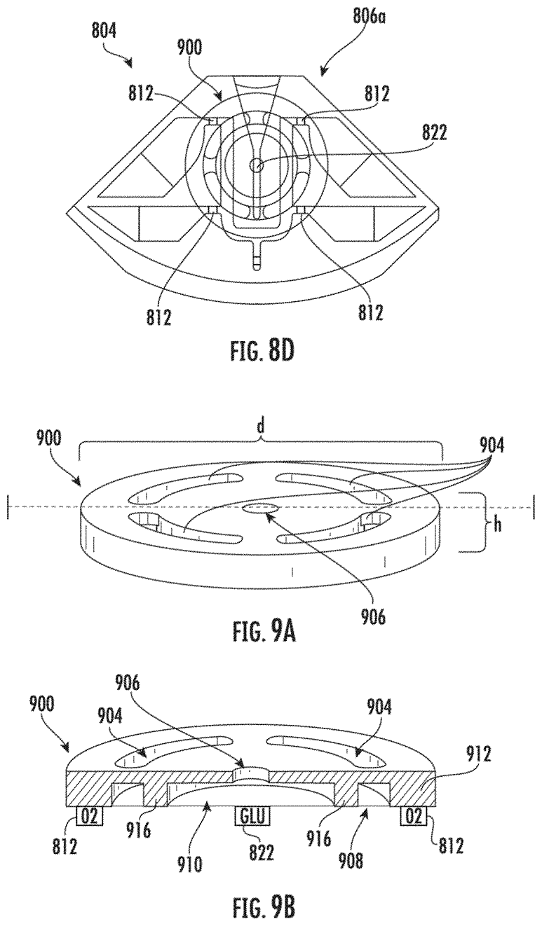

As shown in the exemplary configuration of FIG. 8D, an oxygen permeable membrane structure 900 is disposed on the sensor face 800 over at least a portion of the group of sensing elements 806a of FIG. 8C. Although not specifically shown, it will be appreciated that, in one implementation, each of the groups of sensing elements 806a-806d has a separate and similarly configured membrane structure (such as that shown in FIGS. 8D-9B) associated therewith. In alternate implementations, each group has a separate membrane structure comprising a different configuration (such as e.g., membrane structures each having a different spout size and/or shape) associated therewith. In such implementations, the various sensing element groups may be configured to operate under a different optimal analyte concentration range as defined by e.g., spout diameter and shape, which may regulate analyte diffusion rate toward an active face of the analyte modulated working electrode. In yet another implementation, a single (continuous) membrane structure may substantially cover all of the electrode groups, thereby simplifying and reducing costs of manufacturing for the sensor face. In the latter implementation, the configuration of various areas of the membrane structure each associated with one of the sensing element groups may be similar (e.g., similar spout diameters) or different (e.g., different spout diameters). Various spout membrane configurations usable with the membrane structure 900 are shown and described in U.S. patent application Ser. No. 15/170,571, previously incorporated herein.

In one exemplary embodiment, the sensor elements and face are assembled, such as via method described in U.S. Pat. Nos. 7,894,870 and 8,763,245, each previously incorporated herein, and the pre-formed membrane structure 900 is attached to the sensor face in a specified location (such as that shown in FIG. 8D) with an adhesive such as e.g., silicone. Specifically, an adhesive is applied to one face of the membrane structure which is then adhered to the sensor face, thereby forming a bonded-face of the membrane structure. For example, the membrane structure may be formed and attached to the sensor face via the methods described in U.S. patent application Ser. Nos. 15/170,571 and 15/359,406, each previously incorporated herein.

Turning now to FIGS. 9A and 9B, the membrane structure 900 (e.g., a silicone membrane structure) of FIG. 8D is shown and described in greater detail. As illustrated, the membrane structure 900 includes a substantially cylindrical and planar (i.e., disc-shaped) body of diameter d (in this embodiment, on the order of 0.16 inch) and a height h (which is variable depending on desired response characteristics of the sensing element group). As can be seen in FIG. 9A, the exterior (i.e., non-bonded) face of the membrane structure comprises four outer (peripheral) openings 904 and a central opening (i.e., spout) 906. As can be seen in FIG. 9B, each of the four outer openings 904 communicates with a common channel 908 formed within the membrane structure, while the central opening 906 communicates with a central chamber (or cavity) 910 within the membrane structure.

The central chamber 910 is, in one embodiment, configured to be filled with enzymes (e.g., glucose oxidase (GOX) and catalase enzymes immobilized within a cross-linked albumin (i.e., an “enzyme-embedded” membrane)), and aligned with an active face of the GM working electrode 822 (shown in FIG. 8C). Due to the reaction between glucose and oxygen in the presence of GOX, the glucose electrode senses glucose-modulated oxygen (discussed infra). The central opening or spout 906 is, in one variant, configured to have cross-linked albumin or another such material disposed therein, thereby forming an exterior “enzyme-free” membrane, which retains or limits outward diffusion of reaction by-products (such as e.g., hydrogen peroxide) from the enzyme-embedded membrane and the central chamber 910. Outward diffusion of hydrogen peroxide is further limited via its consumption in the presence of the catalase enzyme. For example, the central chamber and enzyme-embedded and enzyme-free membranes may have the configurations and methods of formation such as those described in U.S. patent application Ser. Nos. 15/170,571 and 15/359,406, each previously incorporated herein.

The membrane structure 900 further comprises a cylindrical ring 912 (an outer perimeter or wall of the membrane structure) configured to be disposed on active faces of the four BO working electrodes 812 (shown in FIG. 8C), and to enable diffusion of oxygen thereto. The cylindrical silicone ring 912 of the membrane 900 is physically connected to a central “riser” area 916 of the membrane structure (which defines the central chamber 910), thereby creating a single silicone membrane seat associated with the group of sensing elements.

In alternate implementations, one or more electrodes within the group of sensing elements may have a separate membrane associated therewith. For example, the aforementioned silicone ring may be a separate structure from the central riser and central cavity, thereby comprising a first membrane structure associated with the GM working electrode and a second (separate) membrane structure associated with each of the BO working electrodes. In another example, the BO working electrodes can each have a separate (individual) solid silicone membrane disposed over its active face. It will be appreciated that in each of the foregoing examples (including the membrane structure 900 shown in FIGS. 8D-9B), the solid silicone membrane associated with the background oxygen detectors enables simplified manufacturing and/or close spatial proximity of the BO working electrodes to the GM working electrode; however, due to e.g., a faster diffusion rate and/or higher permeability of oxygen through silicone relative to a slower diffusion rate and/or lower permeability of oxygen through cross-linked albumin, one or more temporal mismatch correction or mitigation techniques may be required during signal processing for determination of blood glucose data (discussed in detail infra).

Alternatively, in yet other implementations, a membrane aligned with the BO working electrodes (such as e.g., a single membrane structure associated with multiple ones of the BO working electrodes, or individual membrane structures associated with each BO working electrode) can include a cavity or chamber with non-enzymatic cross-linked protein (e.g., albumin) disposed therein. For example, the chamber associated with the BO working electrodes may be substantially similar in its configuration to the central chamber 910 of the membrane structure 900. In such implementations, the design of the membrane(s) for the main (glucose-modulated) and reference (background oxygen) detectors may be such that the response times of each detector to a change in oxygen level are made to closely match (i.e., membranes having a similar diffusion rate and permeability to oxygen). By such matching of response times, artifactual fluctuations in the sensor-reported glucose level (otherwise owing to temporal mismatch of detector oxygen response times) can be minimized, thereby reducing processing requirements (such as e.g., the temporal mismatch correction techniques discussed infra).

Returning to the membrane embodiment of FIGS. 8D-9B, the aforementioned outer periphery openings 904 and the channel 908 of the membrane structure are configured to have one or more biocompatible materials (or any combination of materials), such as crosslinked albumin, silicone, ceramic, metallic materials, etc., or mixtures thereof disposed therein. In one implementation, the outer periphery openings 904 are filled with an oxygen impermeable material (e.g., ceramic, a metallic material, etc.), and the oxygen impermeable material limits diffusion of oxygen from an area of the central chamber 910 to the active face of the BO working electrodes. In such an implementation, cross-talk between the BO working electrodes an the GM working electrode may be physically limited, as outward diffusion of oxygen (e.g., diffusion away from the background oxygen sensing elements) is at least partially blocked.

In other implementations, the outer periphery openings 904 are filled with an oxygen permeable material (e.g., cross-linked albumin, silicone, etc.) or the membrane structure 900 excludes the outer periphery openings and the channel, and instead comprises a solid silicone structure between the cylindrical ring 912 and a central riser 916. In these latter implementations, due to (i) the close proximity between the GM working electrode and the BO working electrodes (discussed supra), and (ii) the absence of an oxygen diffusion barrier between the silicone ring and the central riser, the oxygen concentration at the silicone ring may be influenced by the relative amount of glucose concentration at the central chamber. Therefore, the currents measured by the background oxygen sensing elements tend to exhibit changes pertaining to glucose concentration (deviating from the true background oxygen measurement), a phenomenon which is called “cross-talk.” However, the impact of cross-talk on the glucose computation can be minimized through one or more calibration processes, such as those discussed infra.

### Methods of Operation and Signal Processing

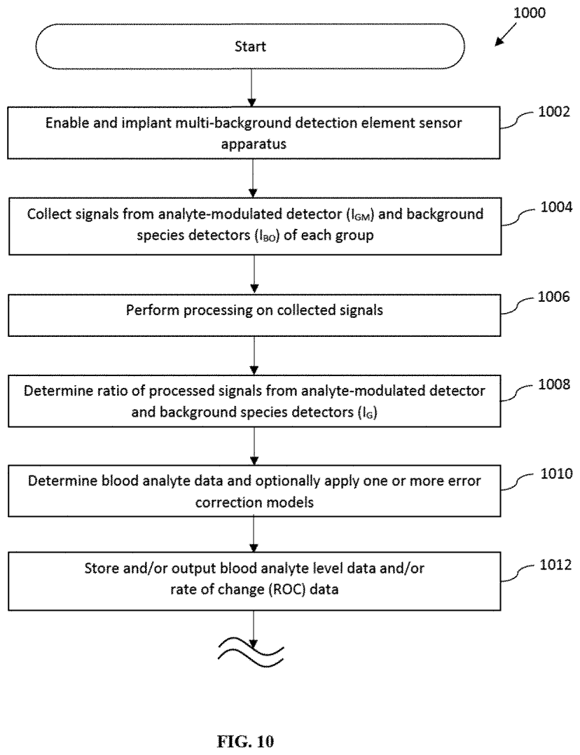

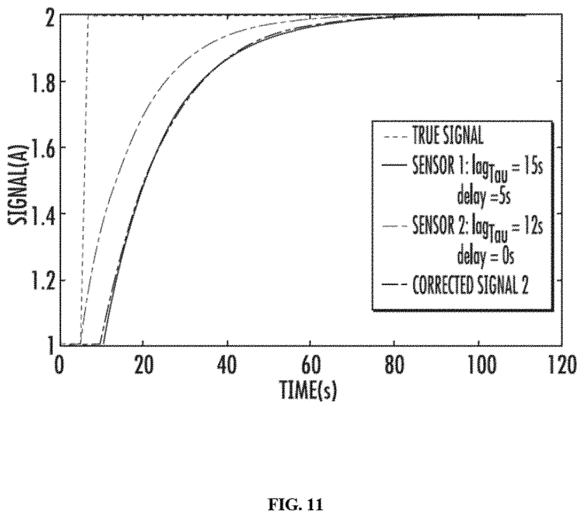

An exemplary generalized method 1000 for operating implantable analyte sensor apparatus disclosed herein is shown in FIG. 10. As depicted therein, at step 1002, an implantable blood glucose sensor apparatus, such as the sensor apparatus 800, is enabled (e.g., powered on, programming initialized, etc.) and implanted in a living subject. In one embodiment, the blood glucose sensor apparatus is implanted in a fully subcutaneous manner, such as via the methods described in U.S. patent application Ser. No. 14/982,346, previously incorporated herein. In an alternate embodiment, the sensor apparatus is only partially implanted; e.g., in a transcutaneous manner.

Next, per step 1004 signals are collected from the differential sensing element groups. For example, as discussed supra, the GM working electrode is in communication and/or operative contact with enzymatic material (e.g., glucose oxidase (GOX) and catalase enzymes immobilized within a cross-linked protein (e.g., albumin) material) disposed within the cavity of the membrane structure. Methods for calculating the levels of glucose present based on a specific enzymatic reaction are well known in the art, as are certain calibration techniques (see, e.g., Choleau, et al., Biosens. Bioelectron., 17:647-654 (2002) and Choleau, et al., Biosens. Bioelectron., 17:641-646 (2002), the teachings of which are incorporated herein by reference in their entirety). Specifically, the exemplary glucose-modulated detector elements described herein in one embodiment utilize the following two-step chemical reaction catalyzed by glucose oxidase and catalase to detect glucose, as described in Armour et al. (Diabetes 39, 1519-1526 (1990)):

Glucose+O₂+H₂O→Gluconate+H₂O₂ (Glucose Oxidase)

H₂O₂→½O₂+H₂O (Catalase)

Glucose+½O₂→Gluconate (Net)

In exemplary embodiments illustrated herein, glucose and ambient oxygen diffuse into the gel matrix and encounter the enzymes, the above reactions occur, and the oxygen that is not consumed in the process is detected by the working or primary electrode.

Note that intervening membrane layers may be included to protect the electrode from drift in sensitivity due to contact with certain non-oxygen chemical species (e.g. electrode “poisoning”), but the detector will nonetheless be arranged sufficiently close to the enzyme-embedded material to enable detection of oxygen levels therein. In embodiments based on “oxygen-sensing differential measurement,” after comparison with the background oxygen concentration detected by one or more of the background species (oxygen) detectors, the difference thereof is related to glucose concentration. In other words, a net reduction or loss of oxygen at the glucose-modulated detector relative to the background oxygen detector is utilized to determine blood glucose data.

The sensor apparatus in such embodiments is therefore minimally composed of (i) the glucose-modulated detector that produces a glucose-modulated, oxygen-dependent current (I_{GM}); (ii) the paired/grouped background oxygen detectors that detect oxygen in the absence of enzymes to produce an oxygen-dependent (glucose-independent) current (I_{BO}); and (iii) a signal-processing element that takes a difference or a ratio of (i) and (ii) to give the signal of interest—the glucose-dependent differential current (I_{G}).

In one implementation, signals are collected from each individual BO sensing element. In another implementation, the four BO sensing elements are physically connected (hardwired, such as in electrical parallel) with each other such that a single measurement is generated (i.e., a total current from the four locations).

When sufficient signals have been collected from the various detector elements, one or more signal processing steps are performed on the signals from the background detector elements and/or the signals from the glucose-modulated detector element for each differential element group (step 1006). In one exemplary implementation of signal processing, the measurements from the four BO electrodes are individually collected and subsequently averaged (via a processor and associated logic) to compute an estimate of the background oxygen for pairing with the glucose-modulated sensing element signal and determination of blood glucose data. Additionally or alternatively, one or more signals from the individual BO sensing elements may themselves be averaged, such as multiple signals from the same sensing element taken at successive times being averaged. This approach may be useful where, for instance, there is significant temporal variability in the signals generated by a given sensing element, and/or latency in response relative to other ones of the elements contributing to the (overall) calculated average.

The averaging may also comprise temporal correlation of signals from each background element (relative to each other, the analyte-modulated element, and/or another sensing element associated with the sensor apparatus); i.e., signals from a common time increment or period are used as the basis of the averaging, such as via the temporal mismatch correction apparatus and methods described in U.S. patent application Ser. No. 16/233,536, previously incorporated herein. As discussed therein (as well as supra), the permeability and diffusion rate of oxygen through the membrane structure to the background species detector elements (e.g., background working electrodes) of the differential group is expected to be greater than that of the analyte-modulated detector element, as permeability of oxygen through the silicone material is greater than the permeability of oxygen through the cross-linked protein material. For example, solubility of oxygen in silicone may be approximately 15 times greater than solubility of oxygen in cross-linked protein, such as e.g., albumin. Accordingly, in such an example, an expected response curve shows the analyte-modulated detector element having higher “delay” and “lag” values than that of a background detector element (where “delay” is related a delay in response of the analyte-modulated detector due to e.g., mechanical and/or material differences in the associated portion of the membrane structure, and “lag” is related to a lag in response of the analyte-modulated detector relative to the background species detector in the presence of analyte). The delay and lag values can be experimentally characterized in vitro or in vivo and applied to sensor signals to correct temporal mismatch such as, e.g., via the apparatus and methods described in U.S. patent application Ser. No. 16/233,536 (previously incorporated herein).

FIG. 11 illustrates one embodiment of the application of one such correction, where a signal from Sensor 2 (e.g., an individual signal from one background element, a composite signal generated by two or more background detector elements, or an averaged from signals from two or more background detector elements) is mathematically transformed so that it more closely matches the response of Sensor 1 (e.g., signals generated by an analyte-modulated detector element) to correct for delay and lag of Sensor 1. Specifically, in this example, the native Sensor 1 signal has a lag_{Tau} of 15 sec and a delay of 5 sec, while the native Sensor 2 signal has a lag_{Tau} of 12 sec and a delay of 0 sec. Via application of a temporal mismatch mitigation or correction algorithm, the Sensor 2 signal is corrected or adjusted to a delay of 5 sec and a lag_{Tau} of 15 sec such that the delay and lag values thereof match those of the Sensor 1 signal.

Notably, the “true” or actual analyte values in FIG. 11 (here modeled as a step response) is only approximated by the Sensor 1 and (transformed) Sensor 2 curves, due to the inherent temporal response (delay and lag) of the detector electrodes. However, by aligning or reconciling the differing temporal responses of the sensor elements, more accurate determination of blood analyte (blood glucose) level is possible, since it is derived from the combined measurements (differential, ratiometric, etc.) of the grouped sensor elements. Stated differently, temporal synchronization of the two (or more) signal responses permits more accurate analyte concentration determination, since the conditions reported by each of the temporally-synchronized sensor elements corresponds (temporally) to the conditions reported by the other sensor element, rather than attempting to compare or utilize one output which is time-delay and/or time-shifted (or time-lagged) from the other in terms of actual physiologic conditions.

It will be appreciated that while the foregoing example applies a transformation of composite or averaged signals from sensor elements with a smaller temporal delay and/or lag (e.g., Sensor 2) in order to temporally align or reconcile with the other electrode having a greater temporal delay and/or lag (e.g., Sensor 1), the various aspects of the present disclosure additionally encompass, inter alia: (i) transformation of the later or more latent signals to “match” the earlier or less latent signals; (ii) application of the aforementioned correction(s) to each of the multiple background species electrodes associated with analyte-modulated electrode (e.g., Sensor 1, Sensor 2, Sensor 3, Sensor 4, and Sensor 5, wherein Sensors 2-5 are reference (background oxygen) electrode signals each with unique or differing temporal response and hence different transformations relative to Sensor 1 of the analyte-modulated sensing electrode); (iii) application of the aforementioned correction(s) to the differential sensor groups (such as e.g., transforming signals from one or more detectors of a first sensor group (e.g., the sensor element group 806a) to temporally correlate with signals from one or more detectors of another sensor group (e.g., the sensor element group 806b); (iv) temporal correlation to different types of electrodes (such as e.g., peroxide-based sensing elements, temperature sensing elements, etc.) included in the sensor apparatus or in signal communication with the sensor apparatus; and/or (v) selective application to only subsets or portions of sensor groups or arrays.

For example, in one implementation, each sensing element group has a separate and similarly configured membrane structure associated therewith (such as, e.g., the membrane structure 900 shown in FIGS. 8D-9B). As the membranes are similarly configured in the foregoing implementation, a response curve for each of the groups of sensing elements is expected to be substantially similar to other groups (i.e., comprising similar delay and lag values). In alternate implementations, each group of sensing elements may have a separate and differently configured membrane structure associated therewith (such as e.g., membrane structures each having a different spout size and shape and/or having a different exterior membrane size and shape disposed within the spout). In such implementations, the sensing element groups may each be configured to operate under a different (at least partially non-overlapping) optimal analyte concentration range as defined by e.g., spout diameter and shape and/or external membrane thereof for regulation of an analyte diffusion rate toward an active face of the analyte-modulated working electrode. For example, various spout and membrane configurations which regulate and/or control diffusion rate are shown and described in co-owned U.S. patent application Ser. No. 15/170,571, previously incorporated herein. In yet another implementation, a single (continuous) membrane structure may substantially cover multiple or all of the electrode groups. In this latter implementation, the configuration of various areas of the membrane structure which are each associated with one of the sensing element groups may be similar (e.g., similar spout diameters and/or shapes) or different (e.g., different spout diameters and/or shapes).

For a sensor face having different or varying membrane configurations (e.g., different spout diameters and/or shapes) associated with the individual sensor groups—whether comprising separate membrane structures or a unitary membrane structure—the expected response curves may vary between the different groups of sensor elements (e.g., an analyte-modulated sensing element may have different delay and/or lag values as compared to analyte-modulated sensing elements of other groups). As each of the differential sensor groups has different response characteristics, the foregoing temporal mismatch correction can advantageously be utilized to correlate signals between the groups.

In another example, a peroxide-based sensing electrode may be additionally utilized in or with the sensor assembly. The glucose signal reported by the peroxide-based electrode of such sensor assembly may have different lag/delay in comparison to the glucose signal reported by the O₂-based sensor (due to, for example, factors inherent in their respective sensing mechanisms, such as, e.g., different enzyme formulations, different membrane structure and/or material properties, etc.). Therefore, the glucose measurements from the two sensor types could be temporally aligned according to the techniques described herein before being combined (by e.g., weighted average, Kalman filter based fusion, etc.) to obtain a more robust estimate.

Notably, in the foregoing temporal correction and averaging strategies, the averaging of the individual sensing element signals may be performed on a per-element basis as well; e.g., only those elements which benefit from such averaging are averaged, whereas other more latent/temporally variable elements or otherwise variant or nonfunctional elements are not included in the average, so as to e.g., reduce processing overhead and sensor energy consumption (via e.g., eliminating or limiting requirements for temporal correction processing) when such processing is performed “on board.” Moreover, individual electrodes exhibiting undesirable or unpredictable behavior (such as excessive noise or signal instability) may simply be eliminated from the averaging (or other) calculation processes. For example, optimization of signal processing at individual sensing elements may be carried out via a training mode operation of the sensor system, similar to the methods and apparatus described in U.S. patent application Ser. Nos. 15/645,913 and 15/853,574, each previously incorporated herein. Specifically, the sensor may be operated in an initial training mode to identify the most effective or “best” background sensing element(s) for use in subsequent normal operation of the sensor apparatus. In such an example, a signal from the identified “best” sensor may be assigned a weight of “1,” whereas others of the background sensing elements are assigned a weight of “0.” Further, a different “best” background sensing element may be selected for use (and assigned a weight of “1”) during subsequent re-training of the sensor.

In yet another exemplary implementation, the currents from the four individual BO sensing elements are combined utilizing computed weights depending on a pre-defined cost or other weighting function. For example, the weights can be computed based on the correlation between signals from each of the BO sensing elements with the signal from the GM sensing element. Such correlations may be in any number of domains, such as e.g., temporal and/or frequency, etc.

In another exemplary signal processing step, the signal data may be corrected for cross-talk caused by diffusion of oxygen to neighboring elements. Specifically, for the BO working electrodes (particularly in embodiments which lack of an oxygen diffusion barrier between the silicone ring and the central riser, such as e.g., those comprising a solid silicone membrane portion encompassing the central cavity), the oxygen concentration may become influenced by the relative amount of glucose concentration at the GM working electrode. For example, at high concentrations of blood glucose, oxygen may diffuse away from the BO working electrodes toward the GM working electrode as the oxygen concentration may be significantly and/or rapidly decreased at the GM working electrode (i.e., within the central cavity) as the enzymatic reaction occurs. Therefore, the currents measured by the background oxygen sensing elements may to exhibit changes pertaining to glucose concentration, which deviate from the true background oxygen measurement (such as, e.g., being lower than the true background oxygen measurement). However, the impact of cross-talk on the glucose computation can be minimized through one or more calibration processes.

In one variant, the differential (ratiometric) current measurement, a ratio of the current from GM electrode with respect to its background oxygen measurement (based e.g., a composite signal or an averaged signal from the BO sensing elements), is approximated as a function (e.g., exponential) of the ratio of true glucose to measured background oxygen (with cross-talk) concentration. Utilizing two or more of the user provided true glucose measurements from a fingerstick meter (or other blood analyte reference data), the corresponding time-matched sensor-reported currents, and background oxygen measurements derived currents from the BO sensor elements allow for the calculation of the exponential function coefficients A and B as shown in Equation 1.

I_{GM}/I_{BO}=A*e^{(B*Cg/pO2)} Equation 1

Specifically, for every fingerstick measurement C_{g,i} collected during a training/calibration period, I_{GM,i} and I_{BO,i} are obtained from the matched sensor sample (i.e., from the measured sensor sample closest in time to the reference data timestamp). Further, the background oxygen measurement pO2ᵢ is calculated by applying factory calibration transform to the I_{BO,i} value. Given the multiple (e.g., four) measurements for each reference measurement, ratios of I_{GM,i}/I_{BO,i} and C_{g,i}/pO2ᵢ are calculated. The values of A and B are computed as those that minimizes a given cost function as shown in Equation 2.

$$arg {min}_{A , B} ( \frac{{I}_{GM}}{{I}_{BO}} - ( A * {e}^{( B * \frac{{C}_{g}}{{pO}_{2}} )} ) \qquad Equation 2$$

The application of the calibrated function to the subsequently measured I_{GM}/I_{BO} signal in an operational mode allows for the computation of C_{g}/pO2 ratio, and consequently C_{g} as the pO2 can be estimated by applying the calibration (scaling) to the I_{BO} signal.

After one or more of the aforedescribed signal and data processing steps, the processed signals are utilized to calculate blood analyte data (such as e.g., blood glucose concentration or level and/or rate of change (ROC)) based at least on the C_{g}/pO₂ ratio, per step 1010.

Also at step 1010 (or alternatively during processing of the collected signals at step 1006), one or more error correction or mitigation models can be utilized in generation of the blood analyte data, and/or applied to the blood analyte data after generation thereof. Methods and apparatus for application of error correction models for improving accuracy blood analyte data are disclosed in U.S. patent application Ser. Nos. 15/645,913 and 15/853,574, each previously incorporated herein. As discussed therein, sensor error due to umodeled system variables (such as e.g., variables which are user physiology and/or context-dependent, and hence may behave differently in each individual and/or context of measurement) can be expressed by the mean absolute relative difference (MARD) between the sensor output and a set of comparison measurements (i.e., a reference measurement), or by the frequency of outliers in the comparison. In one example, the relationship between a measured blood analyte level and a reference blood analyte level (taken at a corresponding point in time) can be expressed by Equation 3 below:

BA_{ref}=BA_{cal}−BAₑᵣᵣₒᵣ−e Equation 3

In Equation 3, “BA_{ref}” is a blood analyte level measured using an external source, “BA_{cal}” is a blood analyte level measured by a calibrated implanted sensor, “BAₑᵣᵣₒᵣ” is systematic error due to unmodeled (and possibly user-specific) system variables, and “e” is error due to random noise.

In one exemplary embodiment, the sensor apparatus employs (i) a training mode of operation, whereby the apparatus (or processing logic associated therewith, whether on-board or off-board on a receiver/processor apparatus or a parent platform apparatus) conducts “machine learning” to model one or more errors (e.g., un-modeled variable system errors) associated with the blood analyte measurement process, and (ii) generation of an operational model (based at least in part on data collected/received in the training mode), which is applied to correct or compensate for the errors during normal operation of the sensor apparatus and generation of blood analyte data.

During operation in the “training mode”, the sensor system collects and calculates time-stamped blood analyte level data (BA_{cal} data), and receives external time-stamped blood analyte level reference data (BA_{ref} data) such as e.g., blood analyte data obtained from “fingersticking”, or other laboratory or in situ testing. The system may additionally collect and utilize other non-BA_{cal} data, such as e.g., data collected from each of the other sensors, non-BA_{cal} data collected from the implanted sensor, and/or data input by a user or medical professional. After collection of a statistically relevant amount of data, the blood analyte reference data and the calculated blood analyte level data are utilized to calculate blood analyte error data (BAₑᵣᵣₒᵣ data), and one or more parameters (e.g., time of day, range of the target blood analyte concentration, temperature, sensor element identification or signal origin (such as, e.g., identification of a first, second, third, and fourth one of the BO elements associated with one of the GM elements, and/or identification of a first, second, third, or fourth one of the differential sensor element groups), heart rate, motion, pressure exerted on the implanted sensor, other blood analyte concentrations, other sensor detector signals or features thereof, such as for example first or second derivatives of sensor signals, or measures of sensor signal variability) which have a high correlation to blood analyte error are identified via application of one or more “machine learning algorithms.” For example, identification of the BO element from which the background oxygen signal originates, identification of the sensor element group from which signal data originates, and/or one or more other variables (such as those listed supra) may be correlated to sensor error, and therefore may be utilized to correct blood analyte data.

Specifically, the error data and data relating to the correlating variables are used in one embodiment to generate a user-specific operational model, which is subsequently used during normal operation of the sensor system in an analyte detection and reporting mode to predict error due to un-modeled system variables (i.e., user and/or context-specific variables), and can be re-trained as necessary. Thus, the output blood analyte level readings advantageously account and/or correct for the predicted un-modeled variable error (and, in some examples, random noise error) over the lifetime of the sensor apparatus, thereby providing significantly improved accuracy in terms of, e.g., mean absolute relative difference (MARD) between the sensor output and a comparison or calibrated measurement, or by the frequency of outliers in such comparisons or calibrations, as compared to conventional implantable blood analyte sensor systems.

Returning to FIG. 10, per step 1012, the generated data is stored at the sensor apparatus and/or output to an external receiver apparatus. For example, the data can be wirelessly transmitted to one or more of a reduced-form, user-wearable receiver apparatus (e.g., wrist worn receiver) or a parent platform (e.g., mobile device) such as those shown and described in U.S. patent application Ser. Nos. 15/368,436 and 16/443,684, each previously incorporated herein.

### Sensor Apparatus

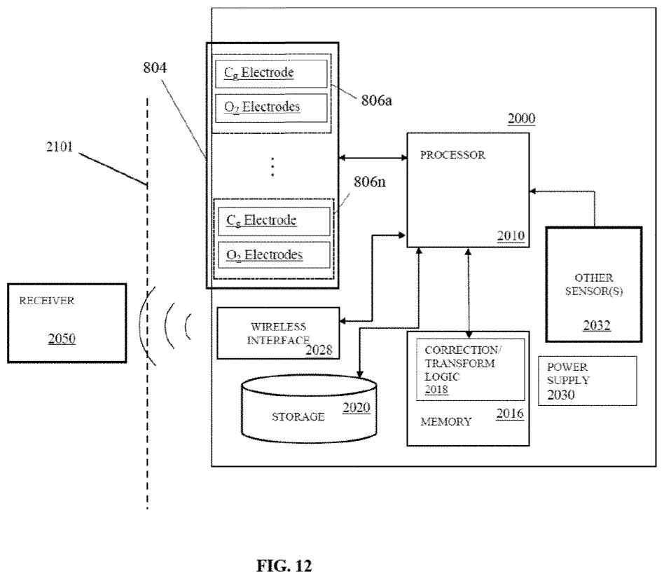

FIG. 12 is a functional block diagram illustrating an exemplary implantable sensor apparatus 2000 and an associated receiver apparatus 2050 according to one embodiment of the present disclosure. As shown, the sensor apparatus 2000 includes a processor 2010 (e.g., digital RISC, CISC, and/or DSP device), and/or a microcontroller (not shown), memory 2016, software/firmware 2018 operative to execute on the processor 2010 and stored in e.g., a program memory portion of the processor 2010 (not shown), or the memory 2016, a mass storage device 2020 (e.g., NAND or NOR flash, SSD, etc. to store collected raw or preprocessed data or other data of interest), a blood glucose element (Cg) and two or more oxygen detector elements (O₂) associated therewith as part of a plurality of differential sensor electrode group 806a-806n, a wireless interface 2028 (e.g., narrowband, PAN such as Bluetooth BLE, 802.15.4 or other) for communication across the interposed tissue barrier 2101, and a power supply 2030 (e.g., a primary Lithium or rechargeable NiMH or Lithium ion battery). Exemplary configurations of sensor and external receiver/parent platforms or devices are described in detail in co-pending U.S. patent application Ser. Nos. 15/853,574, 13/559,475, 14/982,346, 15/170,571, 15/197,104, 15/359,406, 15/368,436, 15/472,091, and 15/645,913, each of the foregoing previously incorporated by reference herein.

Also depicted in FIG. 12, the sensor apparatus 2000 can optionally include one or more additional internal sensors 2032. The internal sensor(s) 2032 may be any of a temperature sensor, an accelerometer, a pressure sensor, a pulse meter, a conductivity meter, pH (i.e., hydronium ion concentration), electric field sensor, and/or other (non-target) analyte-detection sensors (e.g., other blood analytes). In an alternate embodiment, the one or more internal sensors can be located in a separate implantable apparatus positioned proximate to the sensor 2000 during implantation, and may be in data or signal communication with the sensor and/or external receiver or parent platform 2050. As can be appreciated by those of ordinary skill given the present disclosure, any number of different hardware/software/firmware architectures and component arrangements can be utilized for the sensor apparatus 2000 of FIG. 12, the foregoing being merely illustrative. For instance, a less-capable (processing, sensing, and/or data storage-wise) or “thinner” configuration may be used (e.g., excluding the one or more additional internal sensors), or additional functionality not shown added (e.g., including additional types of other sensors and/or components).

In the illustrated embodiment, the logic 2018 is configured to execute on the processor 2010 to implement algorithmic correction (transformation) operations and/or calculation of parameters from data generated by the O₂ detector elements and the Cg detector element, as previously described herein, as well as in U.S. patent application Ser. Nos. 15/645,913; 15/853,574; 16/233,536; and Ser. No. 16/443,684, each of which is previously incorporated by reference herein.

Further, in one embodiment, each of the differential sensor electrode groups 806a-806n has the configuration of the sensor group shown in FIG. 8C, comprising a single glucose-modulated sensing element which is centrally disposed relative to a plurality (e.g., four) associated background oxygen (reference) sensing elements. In alternate embodiments, the glucose-modulated sensing element may be associated with more or background oxygen sensing elements, and/or the electrodes thereof may have a different spatial arrangement. In some implementations, the groups 806a-806n may similar or identical in their arrangement and/or overlying membrane structure (such as to provide duplicative signal data). In other implementations, the groups 806a-806n may be heterogeneous with one another (e.g., each or subsets of groups having a particular membrane/enzyme configuration and/or combination, such as to provide enhanced dynamic range of the sensor overall).

Moreover, the sensor apparatus 2000 of FIG. 12 may be configured in accordance with the various embodiments described in co-owned and co-pending U.S. patent application Ser. Nos. 15/170,571; 15/919,052; 15/359,406; 15/645,913; 15/853,574; 16/233,536; and Ser. No. 16/443,684, each previously incorporated herein, including without limitation the use of on-board blood analyte computation (and model generation), training, and/or power-conserving operating and communication modes.

It will be recognized that while certain embodiments of the present disclosure are described in terms of a specific sequence of steps of a method, these descriptions are only illustrative of the broader methods described herein, and may be modified as required by the particular application. Certain steps may be rendered unnecessary or optional under certain circumstances. Additionally, certain steps or functionality may be added to the disclosed embodiments, or the order of performance of two or more steps permuted. All such variations are considered to be encompassed within the disclosure and claimed herein.

While the above detailed description has shown, described, and pointed out novel features as applied to various embodiments, it will be understood that various omissions, substitutions, and changes in the form and details of the device or process illustrated may be made by those skilled in the art without departing from principles described herein. The foregoing description is of the best mode presently contemplated. This description is in no way meant to be limiting, but rather should be taken as illustrative of the general principles described herein. The scope of the disclosure should be determined with reference to the claims.

What is claimed is:

1. An implantable analyte sensor apparatus configured to determine data related to a concentration of a physiologic analyte of a living being, the implantable analyte sensor apparatus comprising:
at least one group of differential-based detector elements, the at least one group of differential-based detector elements comprising an analyte-modulated detector element and two or more background species detector elements associated therewith, the analyte-modulated detector element and the two or more background species detector elements each at least partially disposed on a sensor face;
data processing apparatus in communication with the at least one differential group of detector elements;
data storage apparatus in data communication with the data processing apparatus, the data storage apparatus having at least one computer program stored thereon, the at least one computer program comprising a plurality of instructions, the plurality of instructions configured to, when executed by the data processing apparatus, cause the implantable analyte sensor apparatus to:
collect first signals generated by the at least one analyte-modulated detector element;
collect second signals generated by the two or more background species detector elements; and
utilize at least data related to the first signals and data related to the second signals to determine at least one of: (i) a concentration of the physiologic analyte, and/or (ii) a rate of change (ROC) of the concentration of the physiologic analyte.

2. The implantable analyte sensor of claim 1, wherein:
the at least one group of differential-based detector elements is disposed in at least a first portion of the sensor face;
the analyte-modulated detector element comprises a first working electrode and a first counter electrode, and each of the two or more background species detector elements comprises a second working electrode and a second counter electrode; and
the analyte-modulated detector element and the two or more background species detector elements are arranged on the sensor face such that: (i) the first working electrode and the second working electrode of each of the two or more background species detector elements are disposed within a central region of the first portion of the sensor face, and (ii) the first counter electrode and the second counter electrode are disposed within a peripheral region of the first portion of the sensor face.

3. The implantable analyte sensor of claim 2, further comprising a membrane structure associated with the at least one group of differential-based detector elements and disposed on the sensor face;
wherein the membrane structure comprises a solid peripheral portion and a cavity, the solid peripheral portion overlying at least the second working electrode of each of the two or more background species detector elements, the cavity overlying at least the first working electrode.

4. The implantable analyte sensor of claim 3, wherein the cavity is disposed at a central region of the membrane structure, the cavity having an enzymatic material disposed therein and being in communication with at least one aperture on an exterior surface of the membrane structure, the at least one aperture having a non-enzymatic cross-linked protein membrane disposed therein, the non-enzymatic cross-linked protein membrane configured to enable diffusion of analyte into the cavity.

5. The implantable analyte sensor of claim 4, wherein:
the membrane structure comprises a material having a higher background species diffusion rate than that of the non-enzymatic cross-linked protein membrane; and
the plurality of instructions are further configured to, when executed by the data processing apparatus, cause the implantable analyte sensor apparatus to:
utilize the first signals to determine data indicative of a response of the at least one analyte-modulated detector element;
utilize the second signals to determine data indicative of a response of the two or more background species detector elements, the data indicative of the response of the two or more background species detector elements having at least one temporal response characteristic different than the response of the at least one analyte-modulated detector element; and
apply a mathematical transformation to temporally correlate the data indicative of a response of the at least one analyte-modulated detector element and the data indicative of the response of the two or more background species detector elements to generate data indicative of temporally correlated background and analyte-modulated responses; and
the utilization of the data related to the first signals and the data related to the second signals to determine the at least one of (i) the concentration of the physiologic analyte, and/or (ii) the rate of change (ROC) of the concentration of the physiologic analyte, comprises utilization of the data indicative of temporally correlated background and analyte-modulated responses to determine the at least one of (i) the concentration of the physiologic analyte, and/or (ii) the rate of change (ROC) of the concentration of the physiologic analyte.

6. The implantable analyte sensor of claim 1, wherein the plurality of instructions are further configured to, when executed by the data processing apparatus, cause the implantable analyte sensor apparatus to:
utilize a cross-talk calibration value to transform at least a portion of the second signals generated by the two or more background species detector elements to generate transformed second signals; and
generate the data related to the second signals utilizing the transformed second signals.

7. The implantable analyte sensor of claim 1, wherein:
the collection of second signals generated by the two or more background species detector elements comprises collection of a composite signal received from the two or more background species detector elements; and
the plurality of instructions are further configured to, when executed by the data processing apparatus, cause the implantable analyte sensor apparatus to generate the data related to the second signals based on the composite signal.

8. The implantable analyte sensor of claim 1, wherein:
the collection of second signals generated by the two or more background species detector elements comprises collection of at least a first signal generated by a first of the two or more background species detectors and a second signal generated by a second of the two or more background species detectors; and
the plurality of instructions are further configured to, when executed by the data processing apparatus, cause the implantable analyte sensor apparatus to generate the data related to the second signals based on an average of at least the first signal and the second signal.

9. The implantable analyte sensor of claim 1, wherein:
the collection of second signals generated by the two or more background species detector elements comprises collection of at least a first signal generated by a first one of the two or more background species detectors and a second signal generated by a second one of the two or more background species detectors; and
the plurality of instructions are further configured to, when executed by the data processing apparatus, cause the implantable analyte sensor apparatus to:
receive external analyte reference data;
evaluate at least the first signal and the second signal to identify one of at least the first signal and the second signal that minimizes error of determined analyte data relative to the external analyte reference data;
generate the data related to the second signals utilizing only the identified one of at least the first signal and the second signal.

10. The implantable analyte sensor of claim 1, wherein:
the two or more background species detector elements comprise (i) a first background species detector element comprising a first working electrode, (ii) a second background species detector element comprising a second working electrode, (iii) a third background species detector element comprising a third working electrode, and (iv) a fourth background species detector element comprising a fourth working electrode; and
the first working electrode, the second working electrode, the third working electrode, and the fourth working electrode are arranged on the sensor face such that a working electrode of the analyte-modulated detector element is centrally disposed therebetween.

11. The implantable analyte sensor of claim 10, further comprising a membrane structure disposed on the sensor face, the membrane structure comprising at least (i) a solid silicone portion overlying each of the first working electrode, the second working electrode, the third working electrode, and the fourth working electrode, and (ii) a central cavity having an enzymatic material disposed therein, the central cavity overlying the working electrode of the analyte-modulated detector element.

12. The implantable analyte sensor of claim 1, wherein the at least one differential group of detector elements comprises at least (i) a first differential group of detector elements having a first membrane structure associated therewith and (ii) a second differential group of detector elements having a second membrane structure associated therewith, the first differential group of detector elements and the first membrane structure configured for analyte detection in a first response range, the second differential group of detector elements and the second membrane structure configured for analyte detection in a second response range, the first response range at least partially non-overlapping with the second response range.

13. An implantable blood glucose sensor apparatus for use in determination of data related to a concentration of blood glucose of a living being, the implantable blood glucose sensor apparatus comprising:
a biocompatible housing; and
a sensing region disposed on a surface of the biocompatible housing, the sensing region comprising:
a first group of differential detector elements, the first group of differential detector elements comprising:
a first glucose-modulated detector element, the first glucose-modulated detector element comprising a first working electrode and a first counter electrode; and
two or more first background oxygen detector elements associated with the first glucose-modulated detector element, each of the two or more first background oxygen detector elements comprising a second working electrode and a second counter electrode; and
at least one membrane structure disposed on at least a portion of the sensor face, the at least one membrane structure comprising:
an interior surface, the interior surface associated with at least the second working electrode of each of the two or more first background oxygen detector elements; and
at least one cavity having an enzymatic material disposed therein, the at least one cavity associated with the first working electrode of the first glucose-modulated detector element;
wherein the first glucose-modulated detector element and the two or more first background oxygen detector elements are configured within the sensing region such that the first working electrode and the second working electrode of each of the two or more first background oxygen detector elements have a prescribed spatial arrangement.

14. The implantable blood glucose sensor apparatus of claim 13, wherein the prescribed spatial arrangement comprises the first glucose-modulated detector element and the two or more first background oxygen detector elements being arranged within the sensing region such that, within the first group of differential detector elements, (i) the first working electrode and the second working electrode of each of the two or more first background oxygen detector elements are centrally disposed relative to the first counter electrode and the second counter electrode of each of the two or more first background oxygen detector elements, and (ii) the first counter electrode and the second counter electrode of each of the two or more first background oxygen detector elements are peripherally disposed relative to the first working electrode and the second working electrode of each of the two or more first background oxygen detector elements.

15. The implantable blood glucose sensor apparatus of claim 13, further comprising a second group of differential detector elements disposed in the sensing region, the second group of differential detector elements comprising a second glucose-modulated detector element and two or more second background oxygen detector elements, the second glucose-modulated detector element comprising at least a third working electrode.

16. The implantable blood glucose sensor apparatus of claim 15, wherein:
the second group of differential detector elements comprises at least one response characteristic that differs from the first group of differential detector elements; and
the at least one membrane structure comprises:
a first membrane structure associated with the first group of differential detector elements, the first membrane structure comprising at least one first aperture disposed on a first exterior surface thereof, the at least one first aperture having a first non-enzymatic membrane disposed therein, one or more of the at least one first aperture or the first non-enzymatic membrane configured to enable diffusion of glucose to the first working electrode at a first rate; and
a second membrane structure associated with the second group of differential detector elements, the second membrane structure comprising at least one second aperture disposed on a second exterior surface thereof, the at least one second aperture having a second non-enzymatic membrane disposed therein, one or more of the at least one second aperture or the second non-enzymatic membrane configured to enable diffusion of glucose to the third working electrode at a second rate, the second rate differing from the first rate.

17. The implantable blood glucose sensor apparatus of claim 15, further comprising:
a third group of differential detector elements disposed in the sensing region, the third group of differential detector elements comprising a third glucose-modulated detector element and two or more third background oxygen detector elements; and
a fourth group of differential detector elements disposed in the sensing region, the fourth group of differential detector elements comprising a fourth glucose-modulated detector element and two or more fourth background oxygen detector elements;
wherein the first group of differential detector elements, the second group of differential detector elements, the third group of differential detector elements, and the fourth group of differential detector elements diverge from a common center of the sensing region in a substantially radial arrangement.

18. A method of operating an implanted analyte sensor apparatus, the implanted analyte sensor apparatus comprising at least one differential sensor element group, the at least one differential sensor element group comprising (i) an analyte-modulated detector element, and (ii) two or more background species detector elements, the method comprising:
collecting first signals generated by the at least one analyte-modulated detector element;
collecting second signals generated by the two or more background species detector elements;
generating data indicative of the response of the at least one analyte-modulated detector element based at least on the collected first signals;
generating data indicative of the response of the two or more background species detector elements based at least on the collected second signals; and
based at least in part on of the data indicative of the response of the at least one analyte-modulated detector element and the data indicative of the response of the two or more background species detector elements, (i) generating one or more of a response ratio and/or a response difference, and (ii) determining blood analyte data for a living being within which the analyte sensor apparatus is implanted.

19. The method of claim 18, further comprising utilizing a cross-talk calibration value to transform at least a portion of the second signals generated by the two or more background species detector elements to generate transformed second signals, the generating the data indicative of the response of the two or more background species detector elements based at least on the transformed second signals.

20. The method of claim 18, further comprising applying a mathematical transformation to temporally correlate the data indicative of a response of the at least one analyte-modulated detector element and the data indicative of the response of the two or more background species detector elements to generate data indicative of temporally correlated background and analyte-modulated responses, the data indicative of temporally correlated background and analyte-modulated response utilized for (i) the generating of the one or more of the response ratio and/or the response difference, and (ii) the determining of the blood analyte data.
